(19) United States
(12) Patent Application Publication
(10) Pub. No.: US 2025/0093684 A1
(43) Pub. Date: Mar. 20, 2025

(54) CIRCADIAN RHYTHM RESTORING BLUE BLOCKERS

(71) Applicant: Arizona Board of Regents on Behalf of the University of Arizona, Tucson, AZ (US)

(72) Inventors: Fabian Fernandez, Tucson, AZ (US); Brooke Jordan Mason, Tucson, AZ (US); Michael Grandner, Tucson, AZ (US); Stanley K. H. Pau, Tucson, AZ (US)

(21) Appl. No.: 18/292,292

(22) PCT Filed: Jul. 25, 2022
(86) PCT No.: PCT/US2022/038207

§ 371 Date: Jan. 25, 2024

Related U.S. Application Data
(60) Provisional application No. 63/225,727, filed on Jul. 26, 2021.

(51) Int. Cl. G02C 7/10; G02B 5/28

(52) U.S. Cl. CPC G02C 7/104; G02B 5/285

(57) ABSTRACT

Methods and devices are described that relate to spectral filters and associated eyewear that are specifically designed to mitigate the disruptive effects of electric lighting on the circadian clock that can cause sleep deprivation and other physiological and psychological maladies. An example wearable device for viewing a real or virtual environment includes one or more windows positioned and a spectral filter that comprises a coating positioned on one or more sections of the one or more windows. The spectral filter includes a multi-layer stack of dielectric material with alternate high and low indices of refraction. The number of the layers and a thickness of each layer are selected to provide designed transmission and blocking characteristics to block circadian-rhythm-disruptive spectra from reaching the wearer's eyes while providing viewability of the real or virtual environment by allowing light outside of the circadian-rhythm-disruptive spectra to reach the wearer's eyes.

### CROSS-REFERENCE TO RELATED APPLICATION(S)

This application claims priority to the provisional application with Ser. No. 63/225,727 titled “CIRCADIAN RHYTHM RESTORING BLUE BLOCKERS,” filed Jul. 26, 2021. The entire contents of the above noted provisional application are incorporated by reference as part of the disclosure of this document.

### TECHNICAL FIELD

The disclosed embodiments relate to eyewear and spectral filters.

### SUMMARY

Methods and devices are described that, among providing other features and benefits, relate to spectral filters and associated eyewear that are specifically designed to mitigate the disruptive effects of electric lighting on the circadian clock that can cause sleep deprivation and other physiological and psychological maladies.

One example wearable device for viewing a real or virtual environment includes one or more windows positioned to allow light from the real or virtual environment to propagate toward a position where a wearer's eyes would be located, and a spectral filter that comprises a coating positioned on one or more sections of the one or more windows. The spectral filter includes a multi-layer stack of dielectric material with alternate high and low indices of refraction such that a layer having a high index of refraction is positioned above or below a layer having a low index of reflection, and a layer having a high index of refraction is positioned above or below a layer having a low index of reflection. The number of the layers and a thickness of each layer are selected to provide designed transmission and blocking characteristics to block circadian-rhythm-disruptive spectra from reaching the wearer's eyes while providing viewability of the real or virtual environment by allowing light outside of the circadian-rhythm-disruptive spectra to reach the wearer's eyes. The designed transmission and blocking characteristics include three contiguous blocking regions at 365-400 nm, 455-495 nm, and 530-560 nm bands of wavelengths, and three contiguous transmission regions at 405-450 nm (±5 nm), 497-528 nm (±2 nm) and 565-695 nm (±5 nm). The spectral filter is configured to block 80-100% of the spectral content in each of the contiguous blocking regions.

### BRIEF DESCRIPTION OF THE DRAWINGS

FIG. 1 illustrates example spectral characteristics of dye-based (left) and pigment-based (right) filters.

FIG. 2A illustrates the solar radiation spectrum.

FIG. 2B illustrates the radiation spectrum of an example light emitting diode (LED).

FIG. 2C illustrates the spectrum of an example halogen lamp.

FIG. 3 illustrates a typical spectral sensitivity of the human eye.

FIG. 4A is a plot of the transmission spectra for a filter specifically designed to restore circadian rhythm in accordance with an example embodiment.

FIG. 4B illustrates a set of parameters associated with the filter of FIG. 4A.

FIG. 5A is a plot of the transmission spectra for a filter specifically designed to restore circadian rhythm in accordance with another example embodiment.

FIG. 5B illustrates a set of parameters associated with the filter of FIG. 5A.

FIG. 6 illustrates an example eyewear that incorporates the circadian rhythm restoring filters of the disclosed technology.

### DETAILED DESCRIPTION

The timing of the brain's circadian clock is set by schedules of natural sunlight exposure. All physiological processes in the body are synchronized to the signals sent from the brain's clock as it interprets these environmental light patterns. As such, the clock's estimation of whether it is daytime (presence of light) or nighttime (near absence of light) has widespread impacts on the timing and organization of the sleep-wake cycle. Electric room lighting can interfere with the circadian clock's estimation of day versus night because the photoreceptors in the eye that send information to the clock are activated by both natural sunlight and electric room lighting. Electric lighting can thus interfere with sleep because humans are most biologically prepared to sleep at night not during the day. If the clock interprets a daytime signal when sensing electric light, it will delay the timing of sleep and will have more difficulty communicating with the rest of the body so that a consolidated period of rest is coordinated at night.

The photoreceptors that transmit light information to the brain's clock are most sensitive to certain parts of the light spectrum. One photoreceptor, melanopsin, is expressed by a subpopulation of cells in the eye called intrinsically photosensitive retinal ganglion cells (ipRGCs). They are most activated by light occurring between 455-495 nm, which is perceived as “blue light.” A second important photoreceptor that relays information to the clock is expressed by cone cells in the retina that are sensitive to mid-wavelength light occurring between 500-560 nm, which is perceived as “green light.” Though blue and green light are only partial pieces of the overall visible spectrum (400-800 nm), they have outsized effects on how the brain's clock measures light exposure and interprets for itself and for the rest of the body the timing and length of the day versus the timing and length of the night.

The circuitry that comprises the brain's clock is different from the circuitry that comprises the image-forming visual system (i.e., the system that allows us to see). Current lens technologies that seek to limit the sleep-disrupting effects of electric lighting at night, take advantage of this segregation by filtering out all incident light below approximately 560 nm and allowing the rest through to enable visibility. This approach blocks about 50% of circadian-active light, while maintaining approximately 80% visibility.

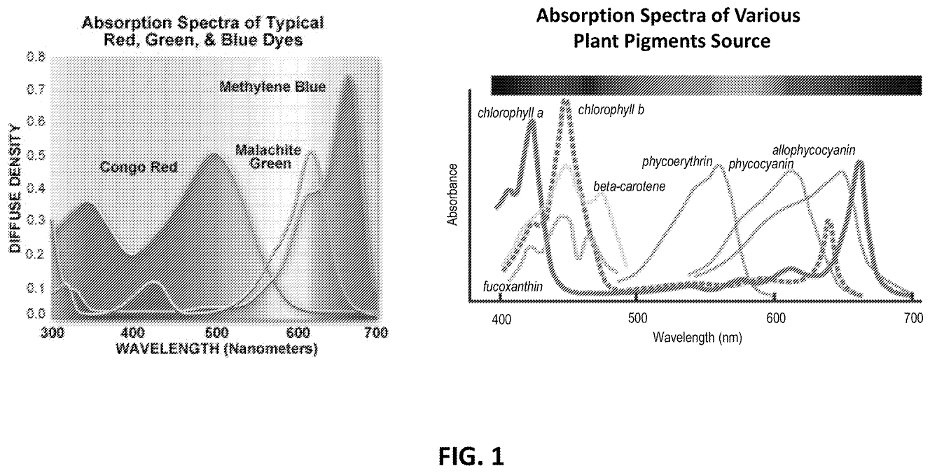

A large number of the existing systems, however, rely on dye- or pigment-based filters that block or transmit a contiguous band of wavelengths but without a capability to selectively transmit or block narrower subbands within the larger contiguous band. Dye and pigment filters operate based on absorption of light by color dye and pigment embedded in a material such as polymer or sol-gel. The transmission spectrum of this type of filter has broad peaks shaped like a Gaussian function with linewidth equal to the inhomogeneous broadening of the materials. FIG. 1 illustrates example spectral characteristics of dye-based (left) and pigment-based (right) filters that exhibit this type of behavior. As also evident from FIG. 1, each spectrum includes a prominent peak with gradual fall off characteristics on both sides thereof. Thus, the usefulness of these filters can be limited to applications where the desired spectral range happens to coincide with the spectral peak of the filter. But even then, the gradual falloff of the spectra can cause part of the desired spectrum to be filtered while allowing part of the undesired spectral content to seep through. In addition, the materials can be bleached under high light intensity, high temperature and/or corrosive environment.

Other types of filters include doped glass, semiconductor, metal, and metamaterial optical filters. Doped glass filters are made of a glass doped with a trace of impurity such as a metal and semiconductor nanocrystal, silver halides and cuprous ions. Semiconductor optical filters are made of semiconductor material with a transmission edge determined by the bandgap. Metal optical filters are made by depositing several layers of metal or metallic alloy made of rhodium, palladium, tungsten, nickel and chromium on a transparent substrate and are used extensively as neutral density filters. Metamaterial optical filters are made of micro- and nano-fabricated structures with dimensions of the order of or smaller than the operating wavelength. Another class of optical filters are tunable optical filters with transmission spectra that can be changed by temperature, electric and/or magnetic field. Examples of tunable optical filters are liquid crystal, Fabry-Perot and MEMS filters. These types of filters are generally bulky and have lower transmission than non-tunable filters.

The disclosed embodiments, among other features and benefits, rely on interference filter designs that are implemented in a wearable device (e.g., glasses, virtual reality goggles, etc.) or used as a covering for a luminaire (e.g., a light source from a house lamp) that provide a more precise and granular spectral behavior in that they allow specific spectral band or bands to be blocked and specific band or bands to be transmitted within a larger spectral bandwidth. The disclosed devices and methods can further allow other spectral bands that are completely blocked by some prior systems to be transmitted. Such a strategy, which would enhance visibility while restricting similar levels of circadian-active light, can be implemented by only blocking the most circadian-active blue and green light occurring between 455-495 and 530-560 nm. The result of this more precise targeting of circadian-active light is that about half the spectrum that is blocked by existing lens technology between 400-560 nm is freed up to improve visibility.

In some embodiments, the spectral content between 365 and 400 nm, the so-called ultraviolet-A (UVA) portion of the light spectrum, is removed. Humans express UVA-sensitive photoreceptors in the eye that could impact retinal function. Though UVA photoreceptors have yet to be definitively implicated in the workings of the brain's circadian clock, they can impact the clock function by impacting how the retina processes circadian-active light at 455-495 nm and 530-560 nm.

The disclosed embodiments rely on multi-layer dielectric interference filter configurations to enable the precise spectral shaping that is required for providing the circadian rhythm restoring characteristics while providing enhanced visibility. Multi-layer dielectric or dichroic filters operate by optical interference instead of absorption. These filters are made by depositing multiple layers of dielectric coating such as magnesium fluoride, zinc sulfide, cerium dioxide, titanium dioxide, silicon oxide, zirconium dioxide to name a few. Interference filters can be designed to transmit light of different wavelength band with sharp transmission edge, in contrast to the broad band spectrum of the dye and pigment filter. The transmission spectrum of this type of filter is generally dependent on the angle of the incident light, although designs can be made to minimize the angular variation.

Several types of interference filters are described that relate to the features of the disclosed embodiments: long-wave pass, short-wave pass, notch (minus, bandstop), and band pass interference filter. A long-wave pass interference filter can include a multilayer structure and can be described using the following shorthand notation:

$${[ \frac{H}{2} L \frac{H}{2} ]}^{S}$$

In the above expression, H denotes a quarter-wave high-index layer having a thickness λ₀/4n_{H} and

$$\frac{H}{2}$$

denotes half of a quarter-wave high-index layer, i.e., one-eighth of a wave λ₀/8 n_{H}. L denotes a quarter-wave low-index layer having a thickness λ₀/4n_{L}; s is an integer that denotes the number of basic periods (i.e., how many times the basis structure of high-low-high is repeated), λ₀ is the reference wavelength (i.e., the center wavelength used to design the filter), and n_{H,L} represents the high or the low refractive index, depending on whether the H or L subscript is used. A short-wave pass interference filter can include a multilayer structure and can be described by the following notation that follows a similar convention as described above:

$${[ \frac{L}{2} H \frac{L}{2} ]}^{S}$$

A bandpass filter is a combination of long-wave pass and short-wave pass filters, and allows only a particular spectral band (i.e., the passband) to be transmitted. A notch filter blocks a particular band of wavelengths (i.e., the notch) but allows the remaining spectral content to pass therethrough. A notch filter can be implemented by using a multilayer structure, represented by the following notation:

$${[ α L β H ]}^{S} α L$$

In the above expression, α and β are numbers chosen for the location and width of the notch filter. For example, a notch filter with a reference wavelength at 550 nm and bandwidth of about 100 nm can be implemented using the multilayer structure represented by:

$${[ 1.68 L 0.3 H ]}^{59} 1.68 L$$

In the above example, α=1.68 and β=0.30.

One key advantage of the disclosed embodiments is the selective transmission and blocking of different wavelengths of light to match the photo receptor sensitivity of the human retina with high efficiency that maintains a high visibility. To this end, the coating on the lens that is part of the eyewear is specifically designed to elicit a particular biological response. To meet these requirements, the optical lens with the coating must satisfy two efficiency conditions: transmission efficiency and illumination efficiency. The transmission efficiency of a color filter can be described as:

$${η}_{T} = \frac{{\int }_{{λ}_{1}}^{{λ}_{2}} d λ T ( λ )}{{\int }_{{λ}_{3}}^{{λ}_{4}} d λ T ( λ )}$$

In the above expression, η_{T} is the transmission efficiency; λ₁ and λ₂ are the lower and upper wavelengths, respectively, of the transmission band; λ₃ and λ₄ are the lower and upper wavelengths, respectively, of the incident illumination; and T(λ) is the filter transmission spectrum. Interference filters with sharp transition edge and low transmission ripple are used to achieve high η_{T}.

In addition, the illumination efficiency of a color filter can be described as:

$${η}_{i} = \frac{{\int }_{{λ}_{1}}^{{λ}_{2}} d λ S ( λ ) T ( λ ) ρ ( λ )}{{\int }_{{λ}_{3}}^{{λ}_{4}} d λ S ( λ ) ρ ( λ )}$$

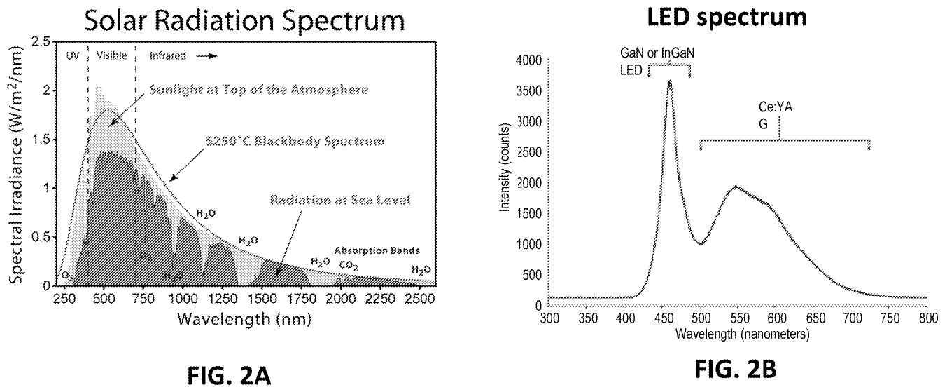

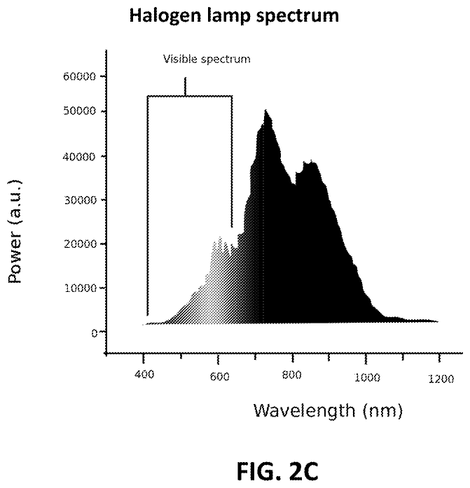

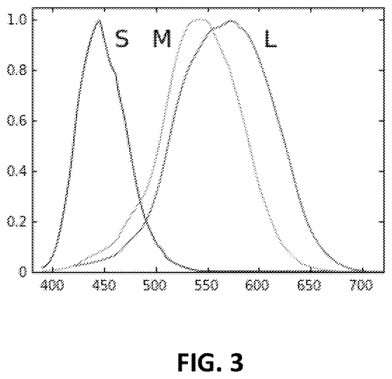

In the above expression, ηᵢ is the illumination efficiency; λ₁ and λ₂ are the lower and upper wavelengths, respectively, of the transmission band; λ₃ and λ₄ are the lower and upper wavelengths, respectively, of the incident illumination; S(λ) is the illumination spectrum; T(λ) is the filter transmission spectrum; and ρ(λ) is the human eye sensitivity. S(λ) can be the spectrum of the sun, a light emitting diode (LED), a halogen lamp, a fluorescent lamp, or another source of illumination. Example spectra of some of the above sources are presented in FIGS. 2A to 2C. In particular, FIG. 2A illustrates the solar radiation spectrum; FIG. 2B illustrates the radiation spectrum of an example LED; and FIG. 2C illustrates the spectrum of an example halogen lamp. FIG. 3 illustrates a typical spectral sensitivity of the human eye, and in particular, the normalized responsivity spectra for S-, M- and L-cone cells. The illumination efficiency of the lens must be high so that light visibility is not critically reduced during day and night.

In some embodiments, the filter is part of a virtual reality and/or augmented reality system. In some embodiments, the filter is designed to be detachable and can be utilized as part of the system during different time of day.

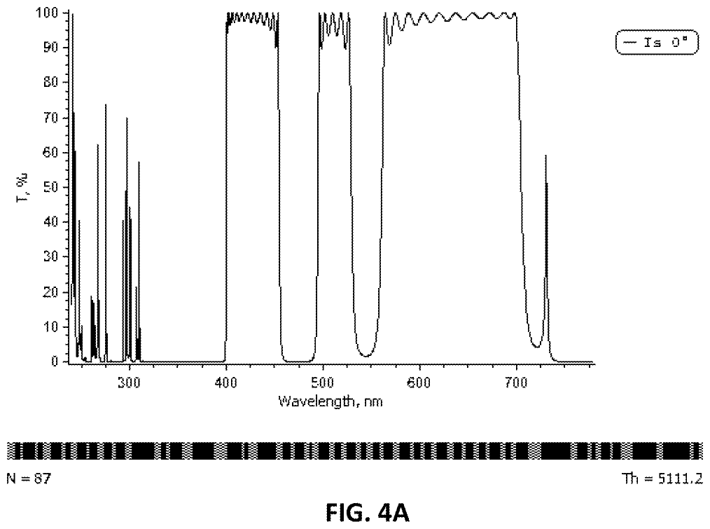

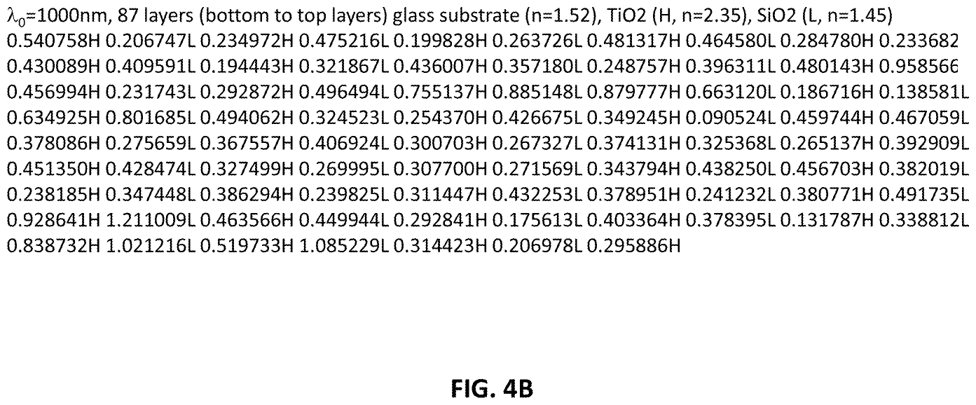

FIG. 4A illustrates a plot of the transmission spectra for a filter specifically designed to restore circadian rhythm in accordance with an example embodiment. The filter in FIG. 4A blocks (i.e., at nearly 0% transmission) the following spectral bands: (1) 365-400 nm, (2) 455-495 nm, and (3) 530-560 nm, and allows very high transmission (in some embodiments, close to 100%) for spectral bands 405-450 nm (±5 nm), 497-528 nm (±2 nm) and 565-695 nm (±5 nm), as well as for the band above 700 nm. It should be also noted that the spectral tolerances (listed in parenthesis) are selected to block all, or almost all, of the green, blue and UV spectral content while allowing as much of the spectral content in the band 500-530 nm to be transmitted. As evident from the plots in FIG. 3, spectral sensitivities of the M and L cone types are highly responsive to the illumination in the 500-530 nm band, and thus illumination in this band improves the visibility and enhances the eye's ability to form images. The filter in FIG. 4A includes 87 dielectric layers stacked on top of a glass substrate (n=1.52), with high and low alternated layers that include TiO₂ (H, n=2.35) and SiO₂ (L, n=1.45), respectively. The thicknesses of the layers (using λ₀=1000 nm as the reference wavelength) are listed in FIG. 4B.

The transmission (or blockage) characteristics of the spectral bands can be modified by changing the number of layers in the design. For example, in some embodiments, the filter is designed to provide a blockage between 98-100%. In other filters, the blockage can be 95-98%. Yet in other designs, the blockage is 80-95%. In some example embodiments, the transmission is in the range 98-100%. Generally, the characteristics of the spectral bands can be fine-tuned by adding more layers to the design at the expense of increasing the cost of the filters.

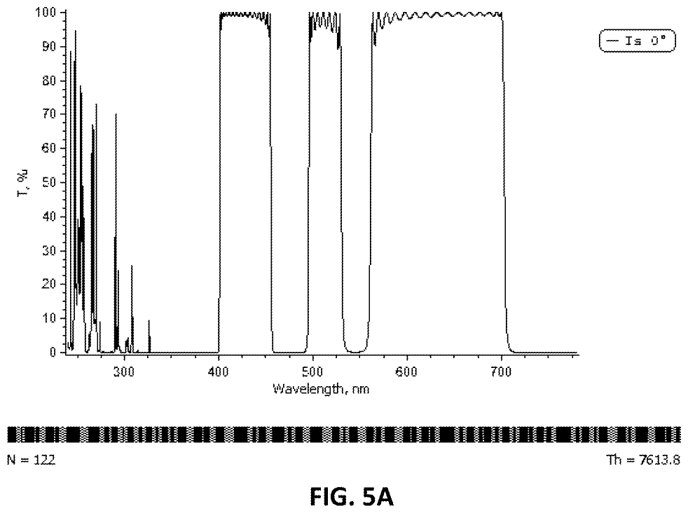

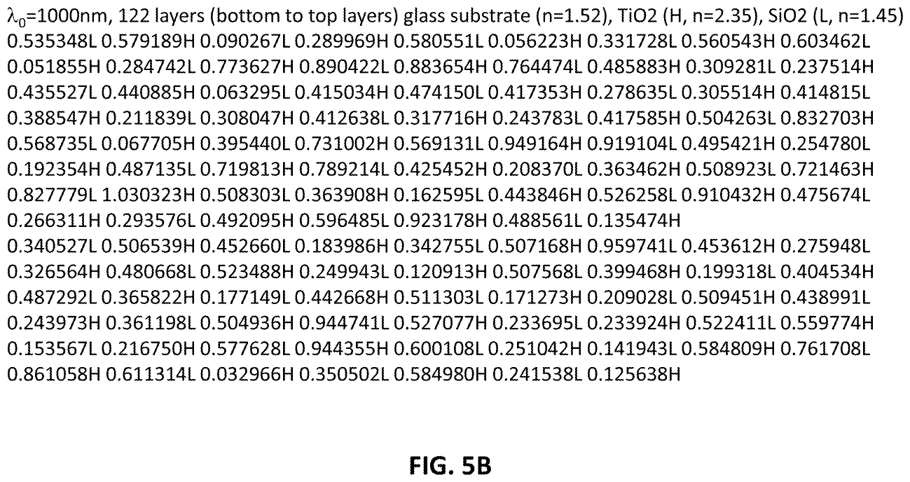

FIG. 5A illustrates a plot of the transmission spectra for a filter specifically designed to restore circadian rhythm in accordance with an alternate embodiment. The filter in FIG. 5, similar to that in FIG. 4, blocks three spectral bands: (1) 365-400 nm, (2) 455-495 nm, and (3) 530-560 nm, and allows nearly full (in some embodiments 100%) transmission for spectral bands 405-450 nm (±5 nm), 497-528 nm (±2 nm) and 565-695 nm (±5 nm). In addition, the FIG. 5 filter blocks nearly all of the spectral ultraviolet spectrum (i.e., content below 400 nm), which has been associated with the incidence of several diseases of the eye. These characteristics are obtained by using 122 dielectric layers stacked on top of a glass substrate with thicknesses that are listed in FIG. 5B. While this design has more layers compared to the design in FIG. 4, it has less ripple and sharper transitions in the transmission spectrum, such that the transmission efficiency for this design is higher. In general, the cost of the filter increases with the number of layers of the filters, and there is a trade-off between cost and transmission efficiency.

Notably, both filters in FIGS. 4A and 5A block the circadian-active blue and green regions occurring in the bands 455-495 nm and 530-560 nm, while allowing nearly 100% transmission in the band 495-530 nm therebetween, and other pass bands such as the band 560-700 nm that are carved out with surgical precision. This added transmission capability in regions where the human eye is spectrally sensitive (see FIG. 3), provides added visibility of the scenes that are being viewed and improves the user experience in wearing the specialized glasses or goggles. In some embodiments, total light transmission is improved by 20-100%, depending on the source of illumination. In one embodiment, the filter allows nearly 50% more irradiance to reach the cornea under cloudy daylight conditions compared to existing designs. In contrast, other types of eyewear that rely on, for example, pigment or dye-based technologies, are often characterized by users to have poor visibility, especially at lowlight conditions such as at night or indoor environments. In addition, both designs in FIGS. 4A and 5A block the UVA region (between 365-400 nm), which as noted earlier, can contribute to the workings of the part of the retina that sends information to the brain's circadian clock. The filters in FIGS. 4A and 5A can also be designed to provide the spectral characteristics for a substrate that is different than glass. The filters may also include an anti-reflection coating on the back side.

Another feature of the disclosed filters is their ability to provide nearly optimum performance for all illumination sources with broadband or relatively broadband spectra. In particular, the filter passbands and notches are designed to precisely coincide with, and have sharp edges, in circadian-sensitive bands. Therefore, they provide optimum performance by only blocking out the portions of the illumination spectra responsible for disruptions in the circadian rhythm.

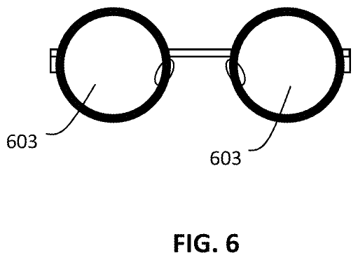

FIG. 6 illustrates a pair of example glasses that includes a pair of lenses 603 that can be coated with the disclosed filters. In some implementations, the glasses can include opaque side shields or blocks (not shown) to prevent side illumination to reach the eye. In some implementations, the side shields can be transparent and include filters with similar transmission and blocking characteristic as those on the lenses 603. As noted earlier, the disclosed filters can be implemented as a coating provided on other types of eyewear, such as virtual reality goggles. For instance, the wearable device can include a unitary transparent window that can be coated uniformly, or at particular locations thereon, with the disclosed filters having transmission and blockage characteristics at precisely tailored bands of the spectrum. For example, the particular locations of coatings can be selected to affect light that reaches the user's eyes at approximately normal angles. In another example, the coatings' locations and areal extent can be chosen to filter the light that reaches the user's eyes at both normal and inclined angles. In yet another example, the filters can be made detachable to existing eye wear, for example by small magnets or by screw-in adapter.

One aspect of the disclosed technology relates to a wearable device for viewing a real or virtual environment that includes one or more windows positioned to allow light from the real or virtual environment to reach a wearer's eyes, and a spectral filter that comprises a coating positioned on one or more sections of the one or more windows. The spectral filter includes a multi-layer stack of dielectric material with alternate high and low indices of refraction such that a layer having a high index of refraction is positioned above or below a layer having a low index of reflection, and a layer having a high index of refraction is positioned above or below a layer having a low index of reflection. The number of the layers and thickness of each layer are selected to provide designed transmission and blocking characteristics to block circadian-rhythm-disruptive spectra from reaching the wearer's eyes while providing viewability of the real or virtual environment by allowing light outside of the circadian-rhythm-disruptive spectra to reach the wearer's eyes. The designed transmission and blocking characteristics include three contiguous blocking regions at 365-400 nm, 455-495 nm, and 530-560 nm bands of wavelengths, and three contiguous transmission regions at 405-450 nm band of wavelengths with a tolerance range of ±5 nm, 497-528 nm band of wavelengths with a tolerance range of ±2 nm and 565-695 nm band of wavelengths with a tolerance range of ±5 nm. The spectral filter is configured to block 80-100% of the spectral content in each of the contiguous blocking regions.

In one example embodiment, each of the contiguous transmission regions transmits 80-100% of the spectral content in the corresponding transmission region. In another example embodiment, the designed transmission and blocking characteristics include a blocking region that extends above 700 nm. In yet another example embodiment, the multi-layer stack includes 87 layers.

In another example embodiment, each layer with the high index of refraction includes titanium dioxide (TiO₂) and has a 2.35 index of refraction, and each layer with the low index of refraction includes silicon dioxide (SiO₂) and has a 1.45 index of refraction. In one example embodiment, the designed transmission and blocking characteristics include a blocking region that extends from 250 nm to 400 nm. In one configuration of the that embodiment, each layer with the high index of refraction includes titanium dioxide (TiO₂) and has a 2.35 index of refraction, and each layer with the low index of refraction includes silicon dioxide (SiO₂) and has a 1.45 index of refraction, and the multilayer stack includes 122 layers.

According to one example embodiment, the one or more windows include two lenses, and the spectral filter is formed as the coating on each of the lenses. In another example embodiment, the wearable device is a pair of goggles, the one or more windows form a unitary window, and the spectral filter is formed as the coating on the unitary window. In still another example embodiment, the wearable device is a pair of goggles, the one or more windows form a unitary window, and the spectral filter is formed as the coating on two or more sections of the unitary window. In yet another example embodiment, the wearable device is a pair of virtual reality goggles.

In another example embodiment, the one or more windows are made of glass or plastic. In one example embodiment, the spectral filter is removably attached to the one or more windows. In yet another example embodiment, the designed transmission and blocking characteristics produce optimum blockage of circadian-rhythm-disruptive light for broadband illumination or ambient lighting conditions.

Another aspect of the disclosed embodiments relates to a spectral filter for use in an eyewear for restoring circadian rhythm that incudes a multi-layer stack coating on a substrate. The multi-layer stack includes a plurality of layers of dielectric material with alternate high and low indices of refraction such that a layer having a high index of refraction is positioned above or below a layer having a low index of reflection, and a layer having a high index of refraction is positioned above or below a layer having a low index of reflection. The number of the layers and a thickness of each layer are selected to provide designed transmission and blocking characteristics to block circadian-rhythm-disruptive spectra from passing through the spectral filter. The designed transmission and blocking characteristics include three contiguous blocking regions at 365-400 nm, 455-495 nm, and 530-560 nm bands of wavelengths, and three contiguous transmission regions at 405-450 nm band of wavelengths with a tolerance range of ±5 nm, 497-528 nm band of wavelengths with a tolerance range of ±2 nm and 565-695 nm band of wavelengths with a tolerance range of ±5 nm. Each of the contiguous blocking regions blocks 80-100% of the spectral content in the corresponding blocking region, and each of the contiguous transmission regions transmits 98-100% of the spectral content in the corresponding transmission region.

In one example embodiment, the designed transmission and blocking characteristics include a blocking region that extends above 700 nm. In another example embodiment, each layer with the high index of refraction includes titanium dioxide (TiO₂) and has a 2.35 index of refraction, and each layer with the low index of refraction includes silicon dioxide (SiO₂) and has a 1.45 index of refraction. In yet another example embodiment, the designed transmission and blocking characteristics include a blocking region that extends from 250 nm to 400 nm. In still another example embodiment, each layer with the high index of refraction includes titanium dioxide (TiO₂) and has a 2.35 index of refraction, and each layer with the low index of refraction includes silicon dioxide (SiO₂) and has a 1.45 index of refraction, and the multilayer stack includes 122 layers. In another example embodiment, the multi-layer stack includes 87 layers.

The foregoing description of embodiments has been presented for purposes of illustration and description. The foregoing description is not intended to be exhaustive or to limit embodiments of the present invention to the precise form disclosed, and modifications and variations are possible in light of the above teachings or may be acquired from practice of various embodiments. The embodiments discussed herein were chosen and described in order to explain the principles and the nature of various embodiments and its practical application to enable one skilled in the art to utilize the present invention in various embodiments and with various modifications as are suited to the particular use contemplated. While operations are depicted in the drawings in a particular order, this should not be understood as requiring that such operations be performed in the particular order shown or in sequential order, or that all illustrated operations be performed, to achieve desirable results. The features of the embodiments described herein may be combined in all possible combinations of methods, apparatus, modules, and systems.

Only a few implementations and examples are described, and other implementations, enhancements and variations can be made based on what is described and illustrated in this patent document.

I/We claim:

1. A wearable device for viewing a real or virtual environment, comprising:
one or more windows positioned to allow light from the real or virtual environment to propagate toward a position of a wearer's eyes; and
a spectral filter that comprises a coating positioned on one or more sections of the one or more windows,
wherein the spectral filter includes a multi-layer stack of dielectric material with alternate high and low indices of refraction such that a layer having a high index of refraction is positioned above or below a layer having a low index of reflection, and a layer having a high index of refraction is positioned above or below a layer having a low index of reflection,
wherein a number of the layers and a thickness of each layer are selected to provide designed transmission and blocking characteristics to block circadian-rhythm-disruptive spectra from passing through the spectral filter while providing viewability of the real or virtual environment by allowing light outside of the circadian-rhythm-disruptive spectra to pass through the spectral filter,
wherein the designed transmission and blocking characteristics include three contiguous blocking regions at 365-400 nm, 455-495 nm, and 530-560 nm bands of wavelengths, and three contiguous transmission regions at 405-450 nm band of wavelengths with a tolerance range of ±5 nm, 497-528 nm band of wavelengths with a tolerance range of ±2 nm and 565-695 nm band of wavelengths with a tolerance range of ±5 nm, and
wherein the spectral filter is configured to block 80-100% of the spectral content in each of the contiguous blocking regions.

2. The wearable device of claim 1, wherein each of the contiguous transmission regions transmits 80-100% of the spectral content in the corresponding transmission region.

3. The wearable device of claim 1, wherein the designed transmission and blocking characteristics include a blocking region that extends above 700 nm.

4. The wearable device of claim 3, wherein the multi-layer stack includes 87 layers.

5. The wearable device of claim 1, wherein:
each layer with the high index of refraction includes titanium dioxide (TiO₂) and has a 2.35 index of refraction, and
each layer with the low index of refraction includes silicon dioxide (SiO₂) and has a 1.45 index of refraction.

6. The wearable device of claim 1, wherein the designed transmission and blocking characteristics include a blocking region that extends from 250 nm to 400 nm.

7. The wearable device of claim 6, wherein each layer with the high index of refraction includes titanium dioxide (TiO₂) and has a 2.35 index of refraction, and each layer with the low index of refraction includes silicon dioxide (SiO₂) and has a 1.45 index of refraction, and the multilayer stack includes 122 layers.

8. The wearable device of claim 1, wherein the one or more windows include two lenses, and the spectral filter is formed as the coating on each of the lenses.

9. The wearable device of claim 1, wherein the wearable device is a pair of goggles, the one or more windows form a unitary window, and the spectral filter is formed as the coating on the unitary window.

10. The wearable device of claim 1, wherein the wearable device is a pair of goggles, the one or more windows form a unitary window, and the spectral filter is formed as the coating on the two or more sections of the unitary window.

11. The wearable device of claim 1, wherein the wearable device is a pair of virtual reality goggles.

12. The wearable device of claim 1, wherein the one or more windows are made of glass or plastic.

13. The wearable device of claim 1, wherein the spectral filter is removably attached to the one or more windows.

14. The wearable device of claim 1, wherein the designed transmission and blocking characteristics produce optimum blockage of circadian-rhythm-disruptive light for broadband illumination or ambient lighting conditions.

15. A spectral filter for use in an eyewear for restoring circadian rhythm, comprising:
a multi-layer stack coating on a substrate, the multi-layer stack including a plurality of layers of dielectric material with alternate high and low indices of refraction such that a layer having a high index of refraction is positioned above or below a layer having a low index of reflection, and a layer having a high index of refraction is positioned above or below a layer having a low index of reflection,
wherein a number of the layers and a thickness of each layer are selected to provide designed transmission and blocking characteristics to block circadian-rhythm-disruptive spectra from passing through the spectral filter,
wherein the designed transmission and blocking characteristics include three contiguous blocking regions at 365-400 nm, 455-495 nm, and 530-560 nm bands of wavelengths, and three contiguous transmission regions at 405-450 nm band of wavelengths with a tolerance range of ±5 nm, 497-528 nm band of wavelengths with a tolerance range of ±2 nm and 565-695 nm band of wavelengths with a tolerance range of ±5 nm,
wherein each of the contiguous blocking regions blocks 80-100% of the spectral content in the corresponding blocking region, and
wherein each of the contiguous transmission regions transmits 98-100% of the spectral content in the corresponding transmission region.

16. The spectral filter of claim 15, wherein the designed transmission and blocking characteristics include a blocking region that extends above 700 nm.

17. The spectral filter of claim 15, wherein each layer with the high index of refraction includes titanium dioxide (TiO₂) and has a 2.35 index of refraction, and each layer with the low index of refraction includes silicon dioxide (SiO₂) and has a 1.45 index of refraction.

18. The spectral filter of claim 15, wherein the designed transmission and blocking characteristics include a blocking region that extends from 250 nm to 400 nm.

19. The spectral filter of claim 18, wherein each layer with the high index of refraction includes titanium dioxide (TiO₂) and has a 2.35 index of refraction, and each layer with the low index of refraction includes silicon dioxide (SiO₂) and has a 1.45 index of refraction, and the multilayer stack includes 122 layers.

20. The spectral filter of claim 15, wherein the designed transmission and blocking characteristics produce optimum blockage of circadian-rhythm-disruptive light for broadband illumination or ambient lighting conditions.

21. The spectral filter of claim 15, wherein the multi-layer stack includes 87 layers.
